(19) United States
(12) Patent Application Publication
(10) Pub. No.: US 2009/0311335 A1
(43) Pub. Date: Dec. 17, 2009

(54) COMBINATION OF A TRIPTAN AND AN NSAID

(76) Inventors: Scott JENKINS, Downingtown, PA (US); Gary LIVERSIDGE, West Chester, PA (US)

Correspondence Address: Fox Rothschild, LLP;Elan Pharma International Limited, 2000 Market Street, Philadelphia, PA, 19103, US

(21) Appl. No.: 12/329,566

(22) Filed: Feb. 24, 2009

Related U.S. Application Data
(60) Provisional application No. 61/061,047, filed on Jun. 12, 2008.

(51) Int. Cl. A61K 9/14; A61P 25/06

(52) U.S. Cl. 424/491

(57) ABSTRACT

A composition of a triptan and particles of a NSAID. The NSAID particles having an effective average particle size of less than 2000 nm and at least one surface stabilizer adsorbed on the surface thereof. The NSAID component of the composition, in a comparative pharmacokinetic testing with a non-particulate NSAID in the same dosage strength and form, exhibits a shorter time to Tₘₐₓ when compared to the time to Tₘₐₓ of the non-nanoparticulate NSAID.

This application claims priority benefit to the U.S. Provisional Application Ser. No. 61/061,047, filed on Jun. 12, 2008.

### BACKGROUND

It has been estimated that 6% of men and 18% of women in the United States currently suffer from migraine headaches. The National Headache Foundation describes characteristics of a migraine headache as including pain typically on one side of the head, pain having a pulsating or throbbing quality, moderate to intense pain affecting daily activities, nausea or vomiting, sensitivity to light or sound, and visual disturbances or aura. Such attacks may last for 4 to 72 hours (sometimes longer). There is currently no test to confirm the diagnosis of a migraine.

Gastric stasis, also referred to as “delayed gastric emptying” or “gastroparesis,” is a common occurrence among migraine sufferers and is manifested by nausea and vomiting. In extreme cases, gastric stasis could cause esophagitis and Mallory-Weiss tear. A consequence of gastric stasis among migraines patients is the slowing down of the disintegration and absorption of the stomach contents which could dramatically impact the pharmacotherapeutic management of such patients. There is a current debate in the literature as to whether gastric stasis appears to be a feature of the disease (migraine attack) or an event that is triggered during an acute migraine attack. See Gastric Stasis in Migraine: More Than Just a Paroxysmal Abnormality During a Migraine Attack. S. K. Aurora, et al.; HEADACHE, January 2006 -Vol. 46 Issue 1 Page 57-63.

The effect a migraine attack has on gastric motility (i.e., gastric stasis) may be approximated in a non-migraine sufferer in the fed state. For example, under fasting conditions, the active phase of digestion occurs every 1 to 2 hours and on average lasts for 5 to 20 minutes, during which the content of the stomach is emptied into the intestine. In the fed state, the gastric motility becomes more intensive and may last continuously for several hours, depending on the size and content of the meal. Peter I. D. Lee & Gordon L. Amidon, Pharmacokinetic Analysis: A Practical Approach (CRC Press 1996). Thus gastric motility in the fed state, when compared to the fasted state, is relatively slower and longer.

A typical therapeutic treatment for a migraine attack is a triptan. Triptans are a family of tryptamine based drugs used as abortive medication in the treatment of migraine and cluster headaches. While effective at treating individual headaches, they are neither a preventative nor a cure. In addition, triptans have been associated with increase gastric stasis. Triptan action is attributed to their binding to serotonin 5-HT₁B and 5-HT_{1D} receptors in cranial blood vessels (causing their constriction) and subsequent inhibition of pro-inflammatory neuropeptide release.

Another typical therapeutic treatment for a migraine attack is an NSAID. An NSAID “Non-steroidal anti-inflammatory drug(s)” are drugs with analgesic, antipyretic and, in higher doses, anti-inflammatory effects—they reduce pain, fever and inflammation. The main adverse drug reaction associated with use of NSAIDs relate to direct and indirect irritation of the gastrointestinal tract (GIT). NSAIDs cause a dual insult on the GIT—the acidic molecules directly irritate the gastric mucosa; and inhibition of COX-1 reduces the levels of protective prostaglandins. Common gastrointestinal adverse drug reactions include nausea/vomiting, dyspepsia, gastric ulceration/bleeding and diarrhea.

U.S. Pat. No. 6,060,499; U.S. Pat. No. 5,872,145; and U.S. Pat. No. 6,384,034 teach various dosages forms containing a combination of a triptan with NSAIDs. These patents are listed in the FDA Orange Book as having claims that cover the commercially available product, TREXIMET®, sold by GlaxoSmithKline of Research Triangle Park, N.C. TREXIMET contains sumatriptan (an exemplary triptan) and naproxen sodium (a water soluble salt form). According to the package insert for TREXIMET®, the sumatriptan T max is about 1 hour and bioavailability of sumatriptan is approximately 15%, partly due to incomplete absorption. The naproxen sodium portion of TREXIMET® has a Tₘₐₓ of about 4 hours with at least a 36% decrease in C max peak with a bioavailability of 95%.

TREXIMET® is an oral tablet containing the above active ingredients and the following inert ingredients: croscarmellose sodium, dextrose monohydrate, dibasic calcium, phosphate, FD&C Blue No. 2, lecithin, magnesium stereate, maltodextrin, microcrystalline cellulose, povidone, sodium bicarbonate, sodium carbosymethylcellulose, talc and titanium dioxide.

The use of conventional formulations combining a triptan and a NSAID for treatment of migraine headaches has shortcomings, e.g., the delayed onset of action for the NSAID portion. This is particularly problematic when the NSAID is used for treating acute migraine headaches where fast pain relief is desirable. Moreover, no conventional formulation combining a triptan and an NSAID has addressed gastric stasis.

### BRIEF DESCRIPTION OF THE DRAWINGS

FIG. 1 is a mean plot the concentration over time of 100 mg nanoKetoprofen (fasted), 100 mg nanoKetoprofen (fed), 50 mg nanoKetoprofen (fasted), 50 mg nanoKetoprofen (fed), 100 mg Orudis (fasted), and 100 mg Orudis (fed).

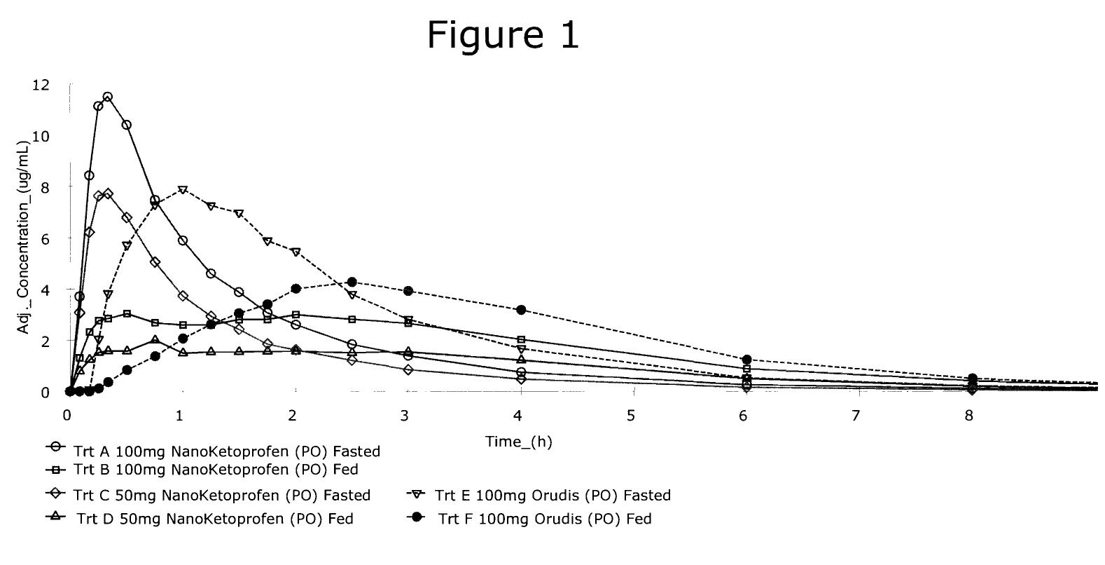

### SUMMARY OF THE INVENTION

A composition of a triptan and particles of a NSAID. The NSAID particles having an effective average particle size of less than 2000 nm and at least one surface stabilizer adsorbed on the surface thereof. The NSAID component of the composition, in a comparative pharmacokinetic testing with a non-particulate NSAID in the same dosage strength and form, exhibits a shorter time to Tₘₐₓ when compared to the time to Tₘₐₓ of the non-nanoparticulate NSAID.

### Detailed Description of the Invention

### DEFINITIONS

As employed above and throughout the disclosure, the following terms, unless otherwise indicated, shall be understood to have the following meanings.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, the term “nanoparticle” or refers to a solid particle of an active agent having a size reported in nanometers (nm) as measured by appropriate methods, for example, sedimentation flow fractionation, photon correlation spectroscopy, light scattering methods, disk centrifugation, or other techniques known to those of skill in the art. When nanoparticles are incorporated into a composition or formulation, such a composition or formulation may be referred to as in “nanoparticulate” form (e.g., a nanoparticulate NSAID means that the NSAID is in nanoparticle form).

Particle size may be determined on a numerical basis or a weight average basis as would be understood by one of ordinary skill in the art.

The population of particles in a given nanoparticulate composition exists as a particle size distribution. Certain features of a particle size distribution are useful to characterize a nanoparticulate composition. As used herein, “effective average particle size” of a particle size distribution means that for a given particle size, x, 50% of the particle population are a size of less than x, and 50% of the particle population are a size that is greater than x. For example, a composition comprising nanoparticles of an NSAID that have an “effective average particle size of 2000 nm” means that 50% of the particles are of a size smaller than about 2000 nm and 50% of the particles are of a size that is larger than 2000 nm.

As used herein, the nomenclature “D” followed by a number, e.g., D₅₀, is the particle size at which 50% of the population of particles in a nanoparticulate composition are smaller and 50% of the population of particles are larger. In another example, the D₉₀ of a particle size distribution is the particle size below which 90% of particles fall, and which conversely, only 10% of the particles are of a larger particle size.

As used herein, a “stable” when used to describe nanoparticles or a nanoparticulate composition connotes, but is not limited to, one or more of the following parameters: (1) the particles do not appreciably flocculate or agglomerate due to interparticle attractive forces or otherwise significantly increase in particle size over time; (2) the physical structure of the particles is not altered over time, (e.g., the morphology of the particles is constant); and/or (3) the particles are chemically stable.

The term “conventional”, “non-nanoparticulate”, or “microparticles” refers to a composition other than a nanoparticulate composition having particle size larger than 2000 nm.

As used herein, the phrase “therapeutically effective amount” means the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “triptan” includes precursors, congeners, salts, complexes, analogs, and derivatives of a triptan. The term “NSAID” includes precursors, congeners, salts, complexes, analogs, and derivatives of an NSAID.

Triptan

Triptans alter the constriction of the blood vessels, which is thought to cause the relief of migraine pain. The triptan is present in the composition in therapeutically effective amounts. It is believed most applications will involve the use of the triptan in an amount of about 0.1 mg to about 200 mg, more likely an amount of about 0.5 mg to about 150 mg, and most likely in an amount of about 1 mg to about 100 mg.

Commercially available triptans include sumatriptan (Imitrex, Imigran), rizatriptan (Maxalt), naratriptan (Amerge, Naramig), zolmitriptan (Zomig), eletriptan (Relpax), almotriptan (Axert, Almogran), and frovatriptan (Frova, Migard).

Exemplary triptans include sumatriptan, rizatriptan, naratriptan, zolmitriptan, eletriptan, almotriptan, and frovatriptan. The triptan within the triptan component can exist in suitable forms, including but not limited to crystalline, amorphous, polymorphs, enantiomers, stereoisomers, and other non-crystalline forms. The triptan can be present in its original crystalline or non-crystalline powder, or further be processed.

NSAID

Nanoparticulate active agent compositions, first described in U.S. Pat. No. 5,145,684 (“the '684 patent”), comprise particles consisting of a poorly soluble therapeutic or diagnostic agent.

NSAIDS inhibit the enzyme responsible for the production of prostaglandins, which are the mediators of pain and inflammation, thereby enhancing the speed, effectiveness and duration of migraine-symptom relief. NSAIDS have traditionally been a reasonable first-line treatment choice for mild to moderate migraine attacks or severe attacks that have been responsive in the past to similar NSAIDS. For example, in a double-blind, placebo-controlled, randomized cross-over trial of a dual-release formulation of oral ketoprofen in the acute treatment of migraine attacks, Dib et al showed that oral ketoprofen (75 mg or 150 mg) in a dual-release formulation is an effective and well-tolerated option. Dib et al, Neurology 2002;58:1660-1665.

The nanoparticulate NSAID of the present invention provides a faster pain relief as compared to the commercially available counterparts in the same dosage strength and form. The NSAID component of the present invention contains suitable NSAIDS in therapeutically effective amounts. The concentration of the NSAID is in an amount of about 0.1 mg to about 1000 mg, about 1 mg to about 800 mg, or about 10 mg to about 600 mg.

Examples of NSAIDS contemplated by the present invention include aspirin, ibuprofen, diclofenac, ketoprofen, pirprofen, naproxen, indomethacin, sulindac, tolmetin, celecoxib, rofecoxib, meclofenamate, mefenamic acid, nambumetone, piroxicam, meloxicam, fenoprofen, flurbiprofen, oxaprozin, etodolac, tolmetin, flurbiprofen, sulindac and ketorolac, loxoprofen and COX-2 inhibitors selected from the group consisting of celecoxib, rofecoxib, valdecoxib, parecoxib, MK-966, etoricoxib, 4-[5-(4-chlorophenyl)-3-(trifluoromethyl)-1H-pyrazol-1-yl)] benzenesulfonamide, N-(2-cyclohexyloxy-4-nitrophenyl)methane sulfonamide, methyl sulfone spiro(2.4)hept-5-ene I, SC-57666, celecoxib, SC-558, SC-560, etodolac, 5,5-dimethyl-3-(3-fluorophenyl)-4-(4-methylsulfonyl)phenyl 2(5H)-furanone, MK-476, L-745337, L-761066, L-761000, L-748780, L-748731, 5-Bromo-2-(4-fluorophenyl)-3-(4-(methylsulfonyl)phenyl, 1-(7-tert.-butyl-2,3-dihydro-3,3-dimethylbenzo(b)furan-5-yl)-4-cyclopropylbutan-1-one, 3-formylamino-7-methylsulfonylamino-6-phenoxy-4H-1-benzopyra-n-4-one, BF 389, PD 136005, PD 142893, PD 145065, flurbiprofen, nimesulide, nabumetone, flosulide, piroxicam, dicofenac, COX-189, D 1367, 4 nitro 2 phenoxymethane sulfonanilide, (3 benzoyldifluoromethane sulfonanilide, diflumidone), JTE-522, 4′-Acetyl-2′-(2,4-difluorophenoxy)m-ethanesulfonanilide, FK 867, FR 115068, GR 253035, RWJ 63556, RWJ 20485, ZK 38997, (E)-(5)-(3,5-di-tert-butyl-4-hydroxybenzylidene)-2-ethyl-1,2-is-othiazolidine-1,1-dioxide indomethacin, CL 1004, RS 57067, RS 104894, SC 41930, SB 205312, SKB 209670, and Ono 1078, their active enantiomers, stereoisomers, analogs and derivatives thereof. A number of the afore-mentioned NSAIDS are currently sold in individually approved, commercially available consumer products.

U.S. Pat. No. 5,518,738 titled “Nanoparticulate NSAID Formulations;” U.S. Pat. No. 5,552,160 titled “Surface Modified NSAID Nanoparticles;” 5,591,456 titled “Milled Naproxen with Hydroxypropyl Cellulose as Dispersion Stabilizer;” U.S. Pat. No. 6,153,225 titled “Injectable Formulations of Nanoparticulate Naproxen;” and U.S. Pat. No. 6,165,506 titled “New Solid Dose Form of Nanoparticulate Naproxen;” and the International Publication WO 1998/35666 exemplify suitable NSAID compositions. Their contents are each incorporated herein by reference.

In an embodiment of the invention, the NSAID is naproxen. Naproxen is a propionic acid derivative ((S)-6-methoxy-methyl-2-naphthaleneacetic acid) which exhibits analgesic and antipyretic properties. Naproxen is often used to relieve the inflammation, swelling, stiffness, and joint pain associated with rheumatoid arthritis, osteoarthritis (the most common form of arthritis), juvenile arthritis, ankylosing spondylitis (spinal arthritis), tendinitis, bursitis, and acute gout. In addition, it is used to treat pain associated with menstrual periods, migraine headaches, and other types of mild to moderate pain. Delivery characteristics and forms are disclosed in, for example, U.S. Pat. Nos. 3,904,682; 4,009,197; 4,780,320; 4,888,178; 4,919,939; 4,940,588; 4,952,402; 5,200,193; 5,354,556; 5,462,747; and 5,480,650, all of which are specifically incorporated by reference in their entirety.

Commercially available naproxen is administered on a two to four times daily basis. Plasma naproxen concentrations of 30-90 μg/ml reportedly are required for anti-inflammatory or analgesic effects. Reduced pain intensity has been demonstrated in sixty postpartum women from 0.5 to 6 hours after oral administration of naproxen in doses sufficient to yield plasma naproxen levels between 30-70 μg/ml. Sevelius, H. et al., Br. J. Clin. Pharmacol. 10, pp. 259-263 (1980). Evidence from twenty-four patients with rheumatoid arthritis suggested that clinical response occurred at plasma naproxen levels above 50 μg/ml. Day, R. O. et al., Clin. Pharmacol, Ther. 31, pp. 733-740 (1982). Thus, while the rate of absorption may affect the onset of analgesic activity, continued plasma levels of the drug are likely to be important in maintaining the analgesia. The present invention provides for improved absorption rates allowing a shorter time to Tmax, thus providing a faster onset of analgesia.

In another embodiment, the NSAID is meloxicam. Meloxicam is an oxicam derivative, also known as 4-hydroxy-2-methyl-N-(5-methyl-2-thiazolyl-)-2-H-1,2-benzothiazine-3-carboxamide 1,1-dioxide, is a member of the enolic acid group of NSAIDs. Meloxicam is practically insoluble in water with higher solubility observed in strong acids and bases. It is very slightly soluble in methanol. The Physicians' Desk Reference, 56th Ed., pp. 1054. Suitable formulations of nanoparticulate meloxicam are described in U.S. Pub. App. 20040229038, the contents of which are incorporated by reference.

Meloxicam exhibits anti-inflammatory, analgesic, and antifebrile activities. Like other NSAIDS, the primary mechanism of action of meloxicam is via inhibition of the cyclooxygenase (COX) enzyme system resulting in decreased prostaglandin synthesis. See The Physicians' Desk Reference, 56th Ed., pp. 1054 (2002). Meloxicam is superior to traditional non-selective NSAIDS because it selectively inhibits COX-2, thus causing fewer gastrointestinal problems such as bleeding, heartburn, reflux, diarrhea, nausea, and abdominal pain. The bioavailability of a single commercial 30 mg oral dose is 89% as compared to a 30 mg intravenous bolus injection. The pharmacokinetics of a single intravenous dose of meloxicam is dose-proportional in the range of 5 to 60 mg. See The Physicians' Desk Reference, 56th Ed., pp. 1054 (2002).

In yet another embodiment, the NSAID is ketoprofen, discussed in more detail in Example 9.

Methods of Making the NSAID Component

Methods of preparing the NSAID component of the formulation are disclosed. NSAID nanoparticulate compositions are prepared by milling the NSAID to obtain a nanoparticulate dispersion comprises dispersing the particles in a liquid dispersion medium in which they are poorly soluble, followed by applying mechanical means in the presence of grinding media to reduce the particle size of the active ingredient to the desired effective average particle size. For example, in case of naproxen, the dispersion medium can be, for example, water, safflower oil, ethanol, t-butanol, glycerin, polyethylene glycol (PEG), hexane, or glycol. A preferred dispersion medium is water. The size of the naproxen particles can be further reduced in the presence of at least one surface stabilizer.

Alternatively, the NSAID particles can be contacted with one or more surface stabilizers after attrition. Other compounds, such as a diluent, can be added to the naproxen/surface stabilizer composition during the size reduction process. Dispersions can be manufactured continuously or in a batch mode.

Another method of forming the desired NSAID nanoparticulate composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the NSAID of choice in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

Another method of preparing the nanoparticulate compositions of the instant invention is by employing a homogenization process. Exemplary homogenization methods of preparing active agent nanoparticulate compositions are described in U.S. Pat. No. 5,510,118, for “Process of Preparing Therapeutic Compositions Containing Nanoparticles.” Such a method comprises dispersing, for example, particles of a naproxen in a liquid dispersion medium, followed by subjecting the dispersion to homogenization to reduce the particle

The population of NSAID particles manufactured by any one of the above-mentioned techniques results in a distribution of NSAID particle of varying size. Certain features of a particle size distribution are useful to characterize a nanoparticulate composition. In an embodiment, the effective average particle size of the NSAID is less 1500 nm, 1400 nm, 1300 nm, 1200 nm, 1100 nm, 1000 nm, 900 nm, about 800 nm, 700 nm, 600 nm, 500 nm, 400 nm, 300 nm, 250 nm, 200 nm, 150 nm, 100 nm, 75 nm, or 50 nm, as measured by appropriate methods known in the art. In another embodiment, the NSAID particle size distribution is characterized by a D₉₀ of less than 2000 nm, 1900, nm, 1800 nm, 1700, nm 1600 nm, 1500 nm, 1400 nm, 1300 nm, 1200 nm, 1100 nm, 1000 nm, 900 nm, 800 nm, 700 nm, 600 nm, 500 nm, 400 nm, 300 nm, 250 nm, 200 nm, 100 nm, 75 nm and 50 nm.

Surface Agent Stabilizers

The surface modifier used must be specifically one which is capable of preventing the agglomeration of NSAID nanoparticles during the milling process of making the nanoparticulae dispersion, and after the dosage form is consumed by a patient. After the dosage form is consumed by a patient, the surface stabilizers must prevent the NSAID particles from aggregating together as the dosage forms dissolves in the GI tract.

Exemplary surface modifiers include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminum silicate, triethanolamine, polyvinyl alcohol, polyvinylpyrrolidone, an ethylene oxide-propylene oxide block copolymer (e.g., poloxamers), dioctylsulfosuccinate, sodium lauryl sulfate, dextran, 4-(1,1,3,3-tetramethylbutyl)-phenol polymer with ethylene oxide and formaldehyde, poloxamines, alkyl aryl polyether sulfonates, mixtures of sucrose stearate and sucrose distearate, p-isononylphenoxypoly-(glycidol), glucamides, glucopuranosides, maltosides, glucosides, PEG-phospholipid, PEG-cholesterol, PEG-cholesterol derivative, PEG-vitamin A, PEG-vitamin E, lysozyme, random copolymers of vinyl pyrrolidone and vinyl acetate, polymers, biopolymers, polysaccharides, cellulosics, alginates, phospholipids, zwitterionic stabilizers, pyridinum compounds, oxonium compounds, halonium compounds, cationic organometallic compounds, quaternary phosphorous compounds, anilinium compounds, ammonium compounds, chitosan, polylysine, polyvinylimidazole, polybrene, polymethylmethacrylate trimethylammoniumbromide bromide (PMMTMABr), hexyldesyltrimethylammonium bromide (HDMAB), polyvinylpyrrolidone-2-dimethylaminoethyl methacrylate dimethyl sulfate, cationic lipids, sulfonium, phosphonium, choline esters, stearalkonium chloride compounds, cetyl pyridinium bromide or chloride, halide salts of quatemized polyoxyethylalkylamines, alkyl pyridinium salts, amines, amine salts, imide azolinium salts, protonated quaternary acrylamides, methylated quaternary polymers, cationic guar, and a carbonium compound.

In embodiments in which the surface modifier is an ammonium compound, the modifier may be a primary ammonium compound, a secondary ammonium compound, a tertiary ammonium compound, or a quarternary ammonium compound. The quarternary ammonium compound may be one of the formula NR˜R˜R˜R4(+) in which:

none of R₁-R₄ is CH₃;

one of R₁-R₄ is CH₃;

three of R₁-R₄ are CH₃;

all of R₁-R₄ are CH₃;

two of R₁-R₄ are CH₃, one of R₁-R₄ is C₆H₅CH₂, and one of R₁-R₄ is an alkyl chain of seven carbon atoms or less;

two of R₁-R₄ are CH₃, one of R₁-R₄ is C₆H CH₂, and one of R₁-R₄ is an alkyl chain of nineteen carbon atoms or more;

two of R₁-R₄ are CH₃ and one of R₁-R₄ is the group C₆H (CH₂)ₙ, where n>1;

two of R₁-R₄ are CH₃, one of R₁-R₄ is C₆H CH₂, and one of R₁-R₄ comprises at least one heteroatom;

two of R₁-R₄ are CH₃, one of R₁-R₄ is C₆H CH₂, and one of R₁-R₄ comprises at least one halogen;

two of R₁-R₄ are CH₃, one of R₁-R₄ is C₆H CH₂, and one of R₁-R₄ comprises at least one cyclic fragment;

two of R₁-R₄ are CH₃ and one of R₁-R₄ is a phenyl ring; or

two of R₁-R₄ are CH₃ and two of R₁-R₄ are purely aliphatic fragments.

Further exemplary surface modifiers include benzalkonium chloride, benzethonium chloride, cetylpyridinium chloride, benztrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyI ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface modifiers are commercially available and/or can be prepared by techniques known in the art. Most of these surface modifiers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000).

The relative amounts of NSAID and surface modifier within the nanoparticle can vary widely. The optimal amount of the individual components can depend, for example, upon the particular NSAID selected, the hydrophilic lipophilic balance (HLB), melting point, and the surface tension of water solutions of the modifier. The concentration of the NSAID within the nanoparticle can vary from about 99.5% to about 0.001%, from about 95% to about 0.1%, or from about 90% to about 0.5%, based on the total combined dry weight of the NSAID and the surface modifier, not including other excipients. The concentration of the surface modifier can vary from about 0.5% to about 99.999%, from about 5.0% to about 99.9%, or from about 10% to about 99.5%, by weight, based on the total combined dry weight of the NSAID and surface modifier, not including other excipients.

Pharmacokinetic Characteristics

In an embodiment, the bioavailability of the NSAID component of a formulation of the invention is improved by a superior showing of such pharmacokinetic parameter as Tmax (i.e., a shorter time to reach maximum concentration) and/or the elimination of the fed/fasted absorption variability of the NSAID. The nanoparticulate NSAID may exhibit a Tₘₐₓ that is not greater than 90%, 80%, 70%, 60%, 50%, 30%, 25%, 20%, 15%, 10%, or 5% of the Tmax for the same non-nanoparticulate NSAID when administered at the same dosage strength and dosage form. For example, an exemplary nanoparticulate naproxen formulation reaches a Tₘₐₓ in nearly half the time as compared to a non-nanoparticulate naproxen in the same dosage form and. Similarly, the time to reach Tₘₐₓ for a 100 mg and 50 mg nanoparticulate ketoprofen formulation is about 50% faster as compared to the non-nanoparticulate commercial counterparts, Orudis®.

Unexpectedly, the nanoparticulate NSAID was even found to exhibit a shorter time to Tmax when compared to a different form of the same NSAID. For example, the nanoparticulate naproxen demonstrated a shorter time to Tmax when compared to a commercially available naproxen sodium (a highly soluble form of naproxen) formulation given at relatively the same dosage strengths.

The NSAID component of the present invention also exhibits a Tₘₐₓ following administration of the composition under fasted conditions that is shorter than that observed for a non-nanoparticulate NSAID administered in the same state. In other embodiments, the nanoparticulate NSAID exhibits a Tmax that is 120 min., 110 min., 100 min., 90 min., 80 min., 70 min., 60 min., 50 min., 40 min., 30 min., 20 min., 15 min., and 10 min. shorter than that observed for a non-nanoparticulate NSAID administered in the same state.

It is known that a common side effect of a migraine headache is nausea. As a result, absorption of the active agent to alleviate the patient's pain should not depend on the patient's stomach contents. In yet another embodiment, the NSAID nanoparticles have no substantial difference in the quantity or rate of absorption when administered to a patient in the fed state versus the fasted state. Eliminating the effect of food may therefore increase patient compliance of migraine sufferers.

The difference in AUC or Cmax of the NSAID when administered in the fed versus the fasted state is less than about 60%, about 55%, about 50%, about 45%, about 40%, about 35%, about 30%, about 25%, about 20%, about 15%, about 10%, about 5%, or about 3%. In one embodiment, the nanoparticulate NSAID administered in the fed state is bioequivalent to the administration of the nanoparticulate NSAID in the fasted state. Under the guidelines of the U.S. Food and Drug Administration, two products or methods are bioequivalent if the 90% confidence intervals for AUC and C max are. between 0.80 and 1.25. Under the guidelines of the European Medicines Agency (EMEA), two products or methods are bioequivalent if the 90% confidence interval for AUC is between 0.80 and 1.25 and the 90% confidence interval for C max is between 0.70 and 1.43.

The relatively bioavailability of the nanoparticulate NSAID when administered to a patient during a migraine attack was about the same compared to when the nanoparticulate NSAID is administered outside of the migraine attack. In other embodiments, the relative bioavailability of the nanoparticulate NSAID when administered to a patient during a migraine attack was 99%, 97%, 95%, 93%, 90%, 87% 85%, 83%, 80%, 77% 75%, 73%, 65%, 60%, 55%, and 50% of the bioavailability of the nanoparticulate NSAID when administered outside of the migraine attack.

In yet another embodiment, within about 5 minutes following administration of the dosage form, at least about 20%, about 30%, or about 40% of the nanoparticulate NSAID is dissolved and made bioavailable. In other embodiments the nanoparticulate NSAID within about 10-20 minutes following administration, at least about 40%, about 50%, about 60%, about 70%, or about 80% of the nanoparticulate NSAID is dissolved. Dissolution is preferably measured in a medium which is predictive of in vivo dissolution of a composition, for example, an aqueous medium containing 0.025M sodium lauryl sulfate. Determination of the amount dissolved can be carried out by spectrophotometry. The rotating blade method (European Pharmacopoeia) may also be used to measure dissolution.

Upon administration of a formulation containing nanoparticles to a subject, the nanoparticles therein may redisperse in vivo. In an embodiment of the present invention, the nanoparticles in the formulation redisperse, following administration thereof to a subject, such that the effective average particle size of the particles is preferably less than about 1500 nm, as measured by appropriate methods, for example, light-scattering methods and microscopy.

In various other embodiments of the present invention, the redispersed nanoparticles have an effective average particle size of less than 1500 nm, 1400 nm, 1300 nm, 1200 nm, 1100 nm, 1000 nm, 900 nm, 800 nm, 700 nm, 600 nm, 500 nm, 400 nm, 300 nm, 250 nm, 200 nm, 150 nm, 100 nm, 75 nm, or 50 nm. In another aspect of the invention, the nanoparticles within the formulation redisperse into same particle sizes as they were originally made prior to their incorporation into the final formulation.

Whether a formulation exhibits the above property may be demonstrated by whether it exhibits this property in biorelevant aqueous media. Such biorelevant aqueous media may be any aqueous media that exhibits ionic strength and pH that are representative of physiological conditions found in the human body. Such media can be, for example, aqueous electrolyte solutions of aqueous solutions of any salt, acid, or base, or a combination thereof, which exhibits the desired pH and ionic strength. Biorelevant pH is well known in the art. For example, in the stomach, the pH ranges from slightly less than 2 (but typically greater than 1) up to 4 or 5. In the small intestine, the pH can range from 4 to 6. In the colon, the pH can range from 6 to 8. Biorelevant ionic strength is also well known in the art. Fasted state gastric fluid has an ionic strength of about 0.1M while fasted state intestinal fluid has an ionic strength of about 0.14M. Appropriate pH and ionic strength values can be obtained through numerous combinations of acids, bases, salts, etc.

Methods of Making the Formulation Comprising a NSAID and a Triptan

The composition of the invention including a nanoparticulate NSAID and a triptan may be made by various methods. Examples of such methods include milling, homogenization, precipitation, freezing, template emulsion techniques, or any combination thereof. The nanoparticulate NSAID, when prepared by the above-described wet milling techniques, is at one step in the process, an aqueous dispersion of nanoparticles which have a surface stabilizer adsorbed on to the surface thereof. The dispersion may be sprayed dried via a fluidized-bed spray dryer granulator into a granulation. The granulation may be combined with other conventional excipients and pressed into minitabs or pellets. Alternatively, the nanoparticle dispersion may be spray-coated onto an inert substrate such as a nonpareil sugar sphere to form beads.

In an embodiment, the triptan component of the formulation is in the form of immediate release beads. By “immediate release”, it is meant that the beads release the triptan immediately upon dissolution of the bead after administration. In an immediate release bead, for example, the nanoparticulate NSAID is spray-coated onto an inert substrate to form a bead, and the triptan is also formulated into an immediate release bead.

A population of the nanoparticulate NSAID beads and a population of the triptan beads are placed into a capsule, which resulting dosage form is referred to in the art as a multiparticulate dosage form. Alternatively, the triptan is formulated into a bead, and the nanoparticulate NSAID is spray-coated onto the triptan bead to form a dual-drug, multi-layered bead. A single population of these dual-drug, multilayered beads may be placed into a capsule for administration to a patient. There are various configurations of the nanoparticulate NSAID and triptan that may be configured according to the desired size, strength and release rate of the NSAID and tiptan components.

For example, in one embodiment, the triptan component is in the form of a modified release bead. By “modified release”, it is meant that the bead allows for a release of the triptan that is not an immediate release.

One exemplary modified release is controlled release. By “controlled release” it is meant that the release of the drug, e.g., the triptan, is characterized by a specific release profile in which, for a specific period of time, a specific rate of release is achieved. Various different rates of release may be achieved at different periods of time. According to an embodiment, the release of the triptan is effectuated by coating the bead of triptan with a controlled release polymer or formulating the triptan into a modified release matrix.

Exemplary controlled release polymers include cellulose acetate phthalate, cellulose acetate trimaletate, hydroxy propyl methylcellulose phthalate, polyvinylacetate phthalate, ammonio methacrylate copolymers such as those sold under the trademark Eudragit® RS and RL, poly acrylic acid and poly acrylate and methacrylate copolymers such as those sold under the trademark Eudragit® S and L, polyvinyl acetaldiethylamino acetate, hydroxypropyl methylcellulose acetate succinate, and shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydoxypropyl cellulose, hydroxypropylmethylcellulose (HPMC), polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate), polyvinylpyrrolidone, anionic and cationic hydrogels, polyvinyl alcohol having, a low acetate residual, a swellable mixture of agar and carboxymethyl cellulose, copolymers of maleic anhydride and styrene, ethylene, propylene or isobutylene, pectin (m. wt. about 30 k-300 k), polysaccharides such as agar, acacia, karaya, tragacanth, algins and guar, polyacrylamides, AquaKeep® acrylate polymers, diesters of polyglucan, crosslinked polyvinyl alcohol and poly N-vinyl-2-pyrrolidone, sodium starch glucolate; hydrophilic polymers such as polysaccharides, methyl cellulose, sodium or calcium carboxymethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageen, guar, xanthan, scleroglucan and mixtures and blends thereof.

In the embodiment where the triptan is formulated in a controlled release matrix, exemplary matrix materials include: hydrophilic polymers, hydrophobic polymers and mixtures thereof which are capable of modifying the release of the compound of interest dispersed therein in vitro or in vivo. Modified-release matrix materials suitable for the practice of the present invention include but are not limited to microcrystalline cellulose, sodium carboxymethylcellulose, hydoxyalkylcelluloses such as hydroxypropylmethylcellulose (HPMC) and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate and mixture thereof.

Another exemplary modified release is a delayed release. By “delayed release” it is meant that the compound is released after a period of delay in which the triptan is not released.

For example, if it is desirable to delay the release of one of the components, an enteric coating may be used. Enteric coatings comprise pH sensitive polymers. Typically, these polymers are carboxylated and interact sparingly with water at low pH. However, at a high pH, the polymer ionizes which causes swelling or the dissolution of the polymers. Such coatings may, therefore, remain intact in the acidic environment of the stomach and then dissolve in the more alkaline environment of the intestine.

The rate and timing of a controlled release formulation of a drug component, e.g., the triptan component, of the present invention may be adjusted by varying the amount of the coating or matrix material, for example, by applying a thicker coating to the particle, or by adjusting the ingredients of the coating or the matrix material.

The dosage forms described above may be combined to form a larger solid dosage form, for example a tablet, a capsule, a lozenge, etc. In one embodiment the triptan and NSAID are co-packaged together. Co-packaging refers to having the dosage forms packaged into the same packaging container (e.g., a blister pack) so that a patient receives a therapeutic dose of NSAID in one tablet/capsule and in the same container a therapeutic dose of the triptan.

Other Ingredients

In addition to the NSAID and the triptan components, the present invention embraces the incorporation of other adjunctive active ingredients in the final formulation, in either nanoparticle or non-nanoparticle forms. Example of such suitable active ingredients include SSRIs such as fluvoxamine, sertaline, fluoxetine; MOA inhibitors such as flenfluramine; antihistamines such as cimetadine or ranitidine; beta blockers such as propranolol; anti-emetics such as metoclopramide, granisetron and ondansetron, anticonvoulsants such as gapapentin; opiates such as hydrocodone and codeine, or other category of drugs generally used in management of migraines or its symptoms, such as nitroglycerine, nimodipine, reserpine, calcium channel blockers, caffeine, ergotamines or combinations thereof.

The formulation of the present invention may comprise also one or more binding agents, filling agents, lubricating agents, suspending agents, sweeteners, flavoring agents, preservatives, buffers, wetting agents, disintegrants, effervescent agents, anti-adherents, and other excipients. Such excipients are known in the art. In embodiments of the present invention which involve the use of particles, including nanoparticles, these excipients may be present within the particle.

In addition, other inactive ingredients could include binding agents, filing agents, lubricants, sweeteners, diluents, disintegrants, preservatives and any other ingredients generally known and preferred by those of ordinary skill in the art.

Examples of binding agents include hydroxypropylmethylcellulose (HPMC).

Examples of filling agents are lactose monohydrate, lactose anhydrous, and various starches.

Examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCCTM). Suitable lubricants, including agents that act on the flowability of the powder to be compressed, are colloidal silicon dioxide, such as Aerosil® 200, talc, stearic acid, magnesium stearate, calcium stearate, and silica gel.—

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; manifold; starch; orbital; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present. Examples of anti-adherents include silicon dioxide and talc.

### EXAMPLES

### Example 1

This example describes the preparation of immediate release particles comprising triptan. Solutions comprising triptan are prepared ((A) to (F)). The formulations are shown in Table 1.

TABLE 1
Triptan Solutions for Immediate Release Particles

| | (A) | (B) | (C) | (D) | (E) | (F) |
|---|---|---|---|---|---|---|
| | Ingredient Amount (percent by weight) | | | | | |
| Naratriptan | 6.0 | 6.0 | 6.0 | 6.0 | 6.0 | 6.0 |
| HPMC 2910 | 1.0 | 2.0 | 2.0 | — | — | 1.5 |
| PEG 600 | — | — | — | 0.5 | — | — |
| Povidone K30 | — | — | — | — | 5.0 | — |
| Fumaric Acid | — | 6.0 | — | — | — | — |
| Citric Acid | — | — | 6.0 | — | — | — |
| Silicon Dioxide | 1.5 | 1.0 | 1.0 | — | — | 2.0 |
| Talc | 1.5 | — | — | — | — | — |
| Purified Water | 90.0 | 85.0 | 85.0 | 93.5 | 89.0 | 90.5 |

Each of these solutions is then coated onto inert sugar spheres (30/35 mesh). The resulting particles have a mean diameter of 0.5 to 0.6 mm.

Hydroxypropylmethylcellulose (HPMC) acts as a binding agent for this coating. Silicon dioxide is an anti-adherent.

### Example 2

This example describes the preparation of modified release triptan containing particles.

Immediate release particles comprising a triptan, such as those prepared in Example 1, are coated with a solution which forms a modified release coating around the particle. Examples of such solutions are provided in Table 2 ((A) to (G)).

TABLE 2
Modified Release Solutions

| | (A) | (B) | (C) | (D) | (E) | (F) | (G) |
|---|---|---|---|---|---|---|---|
| Ingredient | Amount (percent by weight) | | | | | | |
| Eudragit ® RS 100 | 4.1 | 4.9 | 5.5 | | — | 5.5 | 7.5 |
| Eudragit ® RL 100 | — | 1.5 | — | 1.1 | — | — | — |
| Eudragit ® L 100 | 1.4 | — | — | — | — | — | — |
| Ethocel | — | — | — | — | 3.0 | — | — |
| Triethyl Citrate | 1.5 | 1.6 | — | 1.1 | — | — | 1.5 |
| Dibutyl Sebacate | — | — | — | — | 1.6 | 1.0 | — |
| Silicon Dioxide | 1.0 | 1.0 | 1.0 | — | 2.0 | 1.0 | — |
| Talc | 2.5 | 2.5 | 1.0 | 2.8 | — | 1.0 | 2.5 |
| Acetone | 34.0 | 34.0 | 15.0 | 35.6 | — | 14.0 | 33.5 |
| Isopropyl Alcohol | 50.0 | 50.0 | 72.5 | 50.0 | 94.4 | 72.5 | 50.0 |
| Purified Water | 5.5 | 5.5 | 5.0 | 5.0 | — | 5.0 | 5.0 |

Ammonio methacrylate copolymer (Eudragit® RS 100) is a rate-controlling polymer which imparts the controlled-release properties to the particles. Talc is used as an anti-adherent. Acetone and isopropyl alcohol are solvents used in forming a solution of the ammonio methacrylate copolymer. Following the coating of the solution onto the immediate release particle, the solvents evaporate, thus forming a solid coating around the particle. The resulting coated particles are then dried in an oven for about 10 to about 20 hours at about 40 to about 500° C./about 30 to about 60% RH to remove any residual solvents and to obtain a moisture content of about 3 to about 6%.

### Example 3

The purpose of this example is to describe preparation of an ibuprofen nanoparticulate component that can be used in the compositions of the present invention.

Thirty grams of hydroxypropylcellulose (Klucel Type EF; Aqualon) is dissolved in 670 grams of deionized water using a continuous laboratory mixer. The hydroxypropylcellulose serves as a surface modifier. Three hundred grams of ibuprofen is then dispersed into the solution until a homogenous suspension is obtained. A laboratory scale media mill filled with polymeric grinding media is used in a continuous fashion until the mean particle size is approximately 200 nm as measured using a laser light scattering technique.

### Example 4

The purpose of this example is to describe preparation of an ibuprofen nanoparticulate component that can be used in the compositions of the present invention. Twenty five grams of polyvinylpyrrolidone (K29/32; BASF Corp) is dissolved in 575 grams of deionized water using a continuous laboratory mixer.

The polyvinylpyrrolidone serves as a surface modifier. Four hundred grams of ibuprofen is then dispersed into the solution until a homogenous suspension is obtained. A laboratory scale media mill filled with polymeric grinding media is used in a continuous fashion until the mean particle size is approximately 200 nm as measured using a laser light scattering technique.

### Example 5

The purpose of this example is to describe preparation of a naproxen nanoparticulate component that can be used in the compositions of the present invention.

To 575 g of deionized water was dissolved 25 g of polyvinylpyrrolidone (K29/32; BASF Corp) using a continuous laboratory mixer. 400 g of naproxen was dispersed into the PVP solution until a homogenous suspension was obtained. It was processed through a laboratory scale media mill filled with polymeric grinding media in a continuous fashion until the mean particle size was approximately 200 nm as measured by laser light scattering technique, ex. MicroTrak UPA.

### Example 6

The purpose of this example is to describe preparation of a naproxen nanoparticulate component that can be used in the combination compositions of the invention.

A nanoparticulate naproxen dispersion was prepared in a roller mill as follows. A 250 ml glass jar was charged with 120 ml of 1.0 mm pre-cleaned Zirconium oxide beads (Zirbeads XR, available from Zircoa Inc., having a nominal diameter of 1.0 mm), 60 g of an aqueous slurry containing 3 g naproxen (5% by weight), purchased from Sigma, St. Louis, Mo., particle size 20-30 microns, and 1.8 g (3% by weight) Pluronic F-68, purchased from BASF Fine Chemicals, Inc., as the surface stabilizer. The beads were pre-cleaned by rinsing in H₂SO₄ overnight followed by several rinses with deionized water. The batch was rolled at 92 RPM for a total of 120 hours. The dispersion was stable when a portion was added to 0.1N HCl. The average particle size measured by photon correlation spectroscopy was 240-300 nm.

### Example 7

The purpose of this example is to describe methods of preparing meloxicam nanoparticle dispersion. A desired quantity of meloxicam and at least one surface stabilizer can be milled in the presence of suitable rigid grinding media for a suitable period of time in, for example, a DYNO®-Mill KDL (Willy A. Bachofen AG, Maschinenfabrik, Basel, Switzerland), a roller mill (U.S. Stoneware), or a NanoMill® (Elan Drug Delivery Inc.) (see e.g., WO 00/72973 for “Small-Scale Mill and Method Thereof”).

The mean particle size of the resultant compositions, as measured using, for example, a Horiba LA-910 Laser Scattering Particle Size Distribution Analyzer (Horiba Instruments, Irvine, Calif.) is expected to be less than 2 microns. The dispersion is expected to exhibit excellent stability over an extended period of time over a range of temperatures.

### Example 8

The purpose of this example is to describe preparation of a meloxicam nanoparticulate component that can be used in the compositions of the present invention.

The nanoparticulate dispersion of Example 7 can be spray dried, lyophilized, or spray granulated to form a powder. The resulting powder or granules of nanoparticulate meloxicam can then be mixed with the suitable excipients.

Nanoparticulate Meloxicam Spray Dried Powder 50.2 Pregelatinized Starch NF (Colorcon® tarch 20.0 1500) Microcrystalline Cellulose NF (Avicel® PH101) 20.0 Sodium Starch Glycorlate (Explotab®) 5.3 Croscarmellose Sodium USP (Ac-Di-Sol®) 4.0 Magnesium Stearate NF 0.5 Totals 100.0.

The tablets are expected to show excellent redispersion in water as well as in simulated biological fluids. This is significant as redispersion in simulated biological fluids is predictive of redispersion under in vivo conditions.

### Example 9

This example describes the bioavailability of nanoparticulate ketoprofen formulations among patients suffering from acute migraines attack.

In an open-label, single-dose, randomized, fully crossed over, 6 treatment 6 period study with a 5-day washout between treatments. Eighteen (18) healthy volunteers (9 male and 9 female volunteers) aged between 20-37 years and within the following weight range 53.4-88.1 kg were enrolled. Seventeen (17) subjects completed all 6 treatment periods. As migraine attack is commonly associated gastrostasis, in this study the test and reference products were administered under fed conditions in order to mimic this condition among subject patients.

The study comprised of the following 6 different categories:

TABLE 3

| Treatment | | | GI motility |
|---|---|---|---|
| Plan | Dosage | Route | factor |
| A: | NanoKetoprofen 100 mg | orally | fasted |
| B: | NanoKetoprofen 100 mg | orally | fed |
| C: | NanoKetoprofen 50 mg | orally | fasted |
| D: | NanoKetoprofen 50 mg | orally | fed |
| E: | Orudis 100 mg IR | orally | fasted |
| F: | Orudis 100 mg IR | orally | fed |

Human plasma samples were analyzed for ketoprofen levels via HPLC with UV detection at 220 nm (assay range: 0.05-10 ug/mL). This method involved the liquid/liquid extraction of ketoprofen from plasma using diethyl ether. The relative bioavailability for nanoketoprofen 50 mg administered fasted (100.5±22.7%) or fed (86.6±22.5%) were comparable to the administration of the reference product, Orudis 100 mg, fasted.

The range in Cₘₐₓ observed for the test prototypes was as follows: 2.4±2.1 ug/mL (Trt D nanoketoprofen 50 mg)−12.5±3.4 ug/mL (Trt A nanoketoprofen 100 mg). The time to maximum concentration (Tmax) for both nanoparticle formulations, following administration under fasted conditions, was at least one hour shorter than that observed for the reference product administered in the same state.

The Tₘₐₓ observed for both nanoformulations, following administration under fed conditions, were also at least one hour shorter than that observed for the reference product administered in the same state. The t_{1/2} observed for the test prototypes were comparable to that observed for the reference product. The administration of either test prototypes or reference product, under fed conditions, resulted in a decrease in Cₘₐₓ, an increase in Tmax and an extension of the plasma concentration versus time profiles, when compared to the same formulation administered fasted.

In conclusion, the highest bioavailability determined was following fasted administration of 50 mg nanoKetoprofen compared to 100 mg Orudis administered in the same state. All test treatments were well tolerated in this healthy population.

### Example 10

This example describes the bioavailability of nanoparticulate ketoprofen formulations among patients suffering from acute migraines attack.

In another open-label comparative bioavailability study, volunteer patients with prior history of having migraine attacks for at least 12-month were recruited. At least one of the inclusion criteria for entry was experiencing between one and eight moderate or severe attacks per month as defined by the International Headache Society, with or without aura, over at least the previous two months. The qualified subjects were hospitalized for 15 days and underwent a pharmacokinetic sampling study. The subject patients received two single oral administrations of 150 mg of nanoformulation of ketoprofen (one during and one outside a migraine attack).

The pharmacokinetic analysis showed that the mean ketoprofen peak plasma concentration was reduced when the nanoketoprofen was administered to the patients during a migraine attack compared to when administered outside the migraine attack (13.2±6.0 and 18.2±6.6 ug/mL respectively). However, these plasma concentrations were still beyond the required minimum therapeutic levels for migraines treatment.

Tₘₐₓ was prolonged by 1 hr based on median results when the nanoketoprofen was administered to the patients during a migraine attack compared to when administered outside the migraine attack (1.5 h and 0.5 h respectively). These results still suggest faster onset in relation to those of non-nanoparticulate formulations. The relatively bioavailability of nanoketoprofen administered during a migraine attack was 92±17% compared to when administered outside of the migraine attack.

In conclusion, the administration of nanoketoprofen during the course of a migraine attack results in reduced peak concentrations and a delayed time to reach peak concentration compared to administration outside of a migraine attack. However, such results still exceed the levels in comparable non-nanoparticulate formulations. In general, nanoketoprofen was safe and well tolerated in this migraine patient population.

What is claimed is:

1. A composition comprising:
(a) a triptan; and
(b) particles of an NSAID, the particles having an effective average particle size of less than 2000 nm, and at least one surface stabilizer adsorbed on the surface thereof,
wherein in a comparative pharmacokinetic testing with a non-particulate NSAID in the same dosage strength and form, the NSAID having an effective average particle size of less than 2000 nm exhibits a shorter time to Tₘₐₓ when compared to the time to Tₘₐₓ of the non-nanoparticulate NSAID.

2. The composition according to claim 1, wherein the particles of the NSAID are naproxen, and wherein in a comparative pharmacokinetic testing with naproxen sodium in a comparative dosage strength, the nanoparticulate naproxen exhibits a shorter time to Tₘₐₓ when compared to the time to Tₘₐₓ of naproxen sodium.

3. The composition of claim 1, wherein the NSAID is selected form the group consisting of ibuprofen, naproxen, meloxicam, and keotoprofen.

4. The composition of claim 1, wherein when administered to a patient in the fed state, the particles of the NSAID achieve a shorter time to Tmax when compared to the Tmax of a non-particulate NSAID of the same dosage strength administered in the fed state.

5. The composition of claim 1, wherein the Tₘₐₓ of the NSAID when administered to patients during a migraine attack is about 1 hour longer when compared to the Tₘₐₓ of the NSAID when administered to patients outside of a migraine attack.

6. The composition of claim 1, wherein the Tₘₐₓ of the NSAID when administered to patients during a migraine attack is about 1.5 hours and the Tₘₐₓ of the NSAID when administered to patients outside of a migraine attack is about 0.5 hours.

7. The composition of claim 1, wherein the bioavailability of the NSAID when administered to patients during a migraine attack is selected from the group consisting of 99%, 97%, 95%, 93%, 90%, 87% 85%, 83%, 80%, 77% 75%, 73%, 65%, 60%, 55%, and 50% of the bioavailability of the nanoparticulate NSAID when administered outside of the migraine attack.

8. The composition of claim 1, wherein
(i) the triptan is formulated into a bead which comprises an inert substrate overcoated with a layer of the triptan, and
(ii) the NSAID is formulated into a bead which comprises an inert substrate overcoated with a layer of the NSAID particles.

9. The composition of claim 8, wherein the beads of triptan further comprise a rate-controlling polymer overcoating the triptan layer.

10. The composition of claim 8, wherein the pharmacokinetic profile of the composition includes a first drug concentration level spaced apart in time from a second drug concentration level.

11. The composition of claim 10, wherein the first drug concentration level results from the NSAID and the second drug concentration level results from the triptan.

12. The composition of claim 10, wherein the pharmacokinetic profile of the composition includes multiple drug concentration levels, wherein at least one drug concentration level is an NSAID and at least one drug concentration level is the triptan.

13. The composition of claim 1 formulated into a bead which comprises:
(i) an inert substrate,
(ii) a layer of the triptan overcoating the inert substrate, and
(ii) a layer of the NSAID overcoating the triptan layer.

14. The composition of claim 13, wherein the composition is in a multiparticulate capsule dosage form containing a plurality of the beads.

15. The composition of claim 9, wherein a first plurality of triptan beads have a first amount of rate-controlling polymer and a second plurality of triptan beads have a second amount of rate-controlling polymer that is different from the first amount.

16. The composition of claim 8, wherein the composition is in a multiparticulate capsule dosage form containing a plurality of the triptan beads and a plurality of the NSAID beads.

17. The composition according to claim 1, wherein the effective average particle size of the NSAID is selected from the group consisting of less than 1000 nm, of less that 900 nm, less than 800 nm, less than 700 nm, less than 600 nm, less than 500 nm, less than 400 nm, less than 300 nm, less than 250 nm, less than 200 nm, less than 100 nm, less than 75 nm and less than 50 nm.

18. The composition according to claim 1, wherein the particles of the NSAID have a size distribution characterized by a D₉₀ of less than 2000 nm, 1900, nm, 1800 nm, 1700, nm 1600 nm, 1500 nm, 1400 nm, 1300 nm, 1200 nm, 1100 nm, 1000 nm, 900 nm, 800 nm, 700 nm, 600 nm, 500 nm, 400 nm, 300 nm, 250 nm, 200 nm, 100 nm, 75 nm and 50 nm.

19. The composition according to claim 1, wherein the NSAID is present in an amount from about 95% to about 0.1% weight of the total composition.

20. The composition according to claim 1, wherein the surface stabilizer is selected from the group consisting of an anionic surface stabilizer, a cationic surface stabilizer, a zwitterionic surface stabilizer, and an anionic surface stabilizer.

21. The composition according to claim 1, wherein the NSAID is selected from the group consisting of aspirin, ibuprofen, diclofenac, ketoprofen, pirprofen, naproxen, indomethacin, sulindac, tolmetin, celecoxib, rofecoxib, meclofenamate, mefenamic acid, nambumetone, piroxicam, meloxicam, fenoprofen, flurbiprofen, oxaprozin, etodolac, tolmetin, flurbiprofen, sulindac and ketorolac celecoxib, rofecoxib, valdecoxib, parecoxib, MK-966, etoricoxib, 4-[5-(4-chlorophenyl)-3-(trifluoromethyl)-1H-pyrazol-1-yl)] benzenesulfonamide, N-(2-cyclohexyloxy-4-nitrophenyl)methane sulfonamide, methyl sulfone spiro(2.4)hept-5-ene I, SC-57666, celecoxib, SC-558, SC-560, etodolac, 5,5-dimethyl-3-(3-fluorophenyl)-4-(4-methylsulfonyl)phe-nyl 2(5H)-furanone, MK-476, L-745337, L-761066, L-761000, L-748780, L-748731, 5-Bromo-2-(4-fluorophenyl)-3-(4-(methylsulfonyl)phenyl, 1-(7-tert.-butyl-2,3-dihydro-3,3-dimethylbenzo(b)furan-5-yl)-4-cyclopropy-1butan-1-one, 3-formylamino-7-methylsulfonylamino-6-phenoxy-4H-1-benzopyra-n-4-one, BF 389, PD 136005, PD 142893, PD 145065, flurbiprofen, nimesulide, nabumetone, flosulide, piroxicam, dicofenac, COX-189, D 1367, 4 nitro 2 phenoxymethane sulfonanilide, (3 benzoyldifluoromethane sulfonanilide, diflumidone), JTE-522, 4′-Acetyl-2′-(2,4-difluorophenoxy)m-ethanesulfonanilide, FK 867, FR 115068, GR 253035, RWJ 63556, RWJ 20485, ZK 38997, (E)-(5)-(3,5-di-tert-butyl-4-hydroxybenzylidene)-2-ethyl-1,2-is-othiazolidine-1,1-dioxide indomethacin, CL 1004, RS 57067, RS 104894, SC 41930, SB 205312, SKB 209670, and Ono 1078.

22. A method of treating a patient suffering from between one and eight moderate or severe migraine attacks per month comprising administering to the patient the composition of claim 1.

23. A method of treating a patient suffering from between one and eight moderate or severe migraine attacks per month wherein during the attack, the patient presents with gastric stasis comprising administering to the patient the composition of claim 1.

24. A composition comprising:
(a) a first plurality of beads comprising
(i) an inert substrate, and
(ii) a layer of triptan overcoating the inert substrate; and
(b) a second plurality of beads comprising particles of an NSAID having an effective average particle size of less than 2000 nm, at least one surface stabilizer adsorbed on the surface thereof, and exhibiting a shorter time to Tₘₐₓ when compared to the time to Tₘₐₓ of the non-nanoparticulate NSAID,
wherein the pharmacokinetic profile exhibits a first peak of the NSAID spaced apart in time by a second peak of the triptan.

25. The formulation of claim 24, wherein further comprising an active ingredient selected from the group consisting of xanthienes, beta blockers, anti-convulsants, anti-histamines, ergotamines, vasoconstrictors, anti-depressants, and antiemetics.
